(19) United States
(12) Patent Application Publication
(10) Pub. No.: US 2004/0110693 A1
(43) Pub. Date: Jun. 10, 2004

(54) Agent for treating diabetes mellitus

(76) Inventors: Friedrich Trepel, Karlsruhe (DE); Walter Schneider, Arnsberg (DE)

Correspondence Address: Hoffman Wasson & Gitler, Suite 522, 2361 Jefferson Davis Highway, Arlington, VA, 22202, US

(21) Appl. No.: 10/468,409

(22) PCT Filed: Mar. 1, 2002
(86) PCT No.: PCT/EP02/02232

(30) Foreign Application Priority Data
Mar. 2, 2001 (DE) 101 10 124.4
Apr. 5, 2001 (DE) 101 17 185.4
Aug. 2, 2001 (DE) 101 37 459.3

(51) Int. Cl. A61K 31/704; A61K 31/715

(52) U.S. Cl. 514/25; 514/54

(57) ABSTRACT

Composition for the treatment of diabetes mellitus, comprising at least one plant bulk material in combination with at least one alpha-glucosidase inhibitor.

Carbohydrates are a vital plant food constituent. The principal foodstuff is starch (amylose and amylopectin), a polysaccharide constructed from numerous dextrose molecules. Added to this, there are the sugars in the form of disaccharides such as the domestic sugar sucrose, consisting of one molecule of dextrose and one molecule of fructose and finally monosaccharides such as glucose, which only consists of a molecule of dextrose.

Glucose is taken up (absorbed) completely in the small intestine within a few minutes after gastric passage. Sucrose must be cleaved once in the small intestine (to give glucose and fructose) and is therefore available for absorption approximately 10 minutes later. Starch is cleaved many times, the glucose liberated only being absorbed after 20-60 minutes. The cleavage of the carbohydrates is carried out by enzymes (glucosidases and amylases).

The carbohydrates absorbed in the course of the digestive processes are transported to the body cells as “blood sugar” (blood glucose) by the blood circulation in the form of dextrose. After absorption of carbohydrates with the food, in the healthy person the blood sugar increases slightly after ½-1 hour, in the diabetic, however, it increases greatly for many hours. The fasting blood sugar in the healthy person is on average 80-110 mg/dl and increases after a carbohydrate-containing meal to at most 160 mg/dl. In the diabetic, the fasting values vary between 100 and 300 mg/dl. After a meal the blood sugar increases by a further 80-200 mg/dl. A blood sugar concentration of over 160 mg/dl is harmful. It is therefore the aim of any diabetes therapy to lower the blood sugar permanently below this value.

In diabetes (diabetes mellitus), the function of the blood sugar-regulating hormone insulin formed in the pancreas is disturbed. While the healthy person rapidly adjusts again to the normal level of around 100 mg/dl after each carbohydrate meal with the aid of his/her insulin, the diabetic does not succeed in doing this. Two types of diabetes are distinguished. In the type I diabetes beginning in the young person, insulin is completely absent. On the other hand, in the type II diabetes resulting in the second half of life insulin is still formed. However, it has an attenuated and delayed action. In type II diabetes, which makes up over 90% of all diabetics, a gradually decreasing reaction of the body cells to insulin develops prematurely. This insulin resistance leads to an increasing worsening of the course of the disease and should be prevented absolutely by suitable treatment measures. In the affluent countries, diabetes mellitus is the most important people's disease in addition to cardiovascular diseases. In Germany, approximately 6 million people are affected (4 million in the advanced stage, 2 million in the early stage). Worldwide, the number of diabetics may be 200 million. The duration of the disease of type II diabetes is on average 20 years, the disease-related shortening of life approximately 6 years.

Apart from a sugar-free and carbohydrate-controlled diet, five medicinal treatment procedures exist:

1. Insulin injection (in type I obligatory, in type II in the later stages)

2. Antidiabetic tablets of the sulfonylurea type (e.g. glibenclamide) and of the glinide type, which mobilize the remaining insulin reserves in type II diabetes.

3. Antidiabetic tablets of the biguanide type (e.g. metformin), which bring about non-physiological glucose utilization in the type II diabetic and thereby lower the blood sugar.

4. Antidiabetic tablets of the glitazone type, which make the body cells sensitive to insulin again and can therefore have a hypoglycemic action in type II diabetes.

5. Antidiabetic tablets which, by inhibition of the carbohydrate-cleaving glucosidases, reduce the glucose absorption in the small intestine and thus decrease the blood sugar increase after eating. These alpha-glucosidase inhibitors can be used in both types of diabetes.

Status of the Therapy Options

Re 1:

Insulin is the strongest hypoglycemic medicament and represents the basic therapy of type I diabetes which is necessary for survival. In type II, its use is restricted to the later stages, in which the antidiabetic tablets available hitherto no longer have an adequate effect. In addition to its indispensable positive effects, insulin has side effects (life-endangering hypoglycemias, promotion of arterio-sclerosis, promotion of weight increase).

Re 2:

Sulfonylureas and glinides initially have a very good hypoglycemic action, but lose their effect after a few years because of the exhaustion of the insulin stores, promote weight increase and, like insulin, often have severe side effects (e.g. hypoglycemias).

Re 3:

Biguanides have a moderately strong hypoglycemic action, but must not be used in the many patients having diabetic late damage or an age of over 70 years and often have side effects, among them serious ones.

Re 4:

Glitazones are only permitted for therapy in combination with sulfonylurea or biguanide preparations, because their action only commences after 8-12 weeks' treatment and is relatively weak. As an undesired side effect in the type II diabetics in particular, glitazones lead to a weight increase.

Re 5:

The alpha-glucosidase inhibitors do reliably decrease the blood sugar increase after eating, but in approximately half of the patients treated have troublesome side effects on the part of the gastrointestinal tract, so that treatment in an adequate dose is restricted thereby.

The conclusion to be emphasized is: All five therapy principles are effective in type II diabetes, but burdened with considerable side effects substance-specifically (sulfonylureas, biguanides) or in the therapeutically optimum dose (insulins, sulfonylureas, biguanides, alpha-glucosidase inhibitors). The protection of the endogenous insulin reserves necessary as a long-term therapy aim is only achieved by insulin, biguanides and alpha-glucosidase inhibitors. The undesired hyperinsulinemia is only decreased by glitazones, biguanides and alpha-glucosidase inhibitors. The most desirable therapy in the early and middle stages of type II diabetes would in fact be therapy with alpha-glucosidase inhibitors, because it has no dangerous side effects together with medium-grade efficacy, but most patients do not tolerate the troublesome concomitant symptoms (flatulence, proneness to diarrhea).

WO 93/16 605 describes the therapeutic use of indigestible fiber preparations having a proportion of 25-60% of beet bulk material (corresponding to 1.25-12 g/day) for lowering raised blood pressure in humans. The underlying data were compiled on blood pressure patients who did not have diabetes. In the course of comprehensive data acquisition about the kidney and thyroid function and the lipid metabolism of these study participants, the fasting blood sugar and the fasting concentration of insulin in the blood serum and the indirect integral blood sugar measurement HbA1C was also measured. The fasting blood sugar remained unchanged after bulk treatment for a number of months, but the insulin level fell. The HbA1C value decreased in the bulk group just as in the control group. From these results, the authors have derived no clear effect of (beet) bulk material on the carbohydrate metabolism of nondiabetics.

Bulk materials were introduced into diabetes therapy 20 years ago. Bulk materials are nondigestible plant substances which accompany the food. Until now, especially water-soluble bulk materials such as guar, pectin, psyllium and beta-glucans proved effective. In an adequate dose these substances, however, all have therapy-restricting properties such as disturbances of the gastric activity or taste and swallowing impairments. In contrast to the soluble bulk materials, the water-insoluble bulk materials were not systematically investigated in the therapy of human diabetes until now. Water-insoluble bulk materials are the polysaccharides cellulose, hemicellulose (pentosans) and the phenylpropane polymer lignin.

The present invention describes ways in which the blood sugar increase after eating is lowered reliably, greatly and without relevant side effects or concomitant symptoms and long-term improvements in the course of the disease are achieved.

It is the object of the present invention to make available a composition for the treatment of diabetes mellitus which has a good efficacy without the abovementioned side effects occurring.

This aim is achieved by a composition for the treatment of diabetes mellitus having the features of one of patent claims 1, 6, 22, 24 or 33.

The composition according to the invention for the treatment of diabetes mellitus is distinguished according to a first variant of the invention in that it contains beet bulk material as an active compound. The composition according to the invention is preferably employed for the treatment of the early stages of diabetes mellitus type II.

Beet bulk material is fine-grain chips (granules) of sugar beet residues, the “sugar beet pulp”, after previous recovery or separation of the sugar.

Its composition is, for example, pectin 31%, pentosans 24%, cellulose 24%, lignin 4%, protein 9%, ash (mineral salts) 4%, sucrose 4% (average values). Sugar beet residues of this type serve especially as a livestock feed additive. In the form of the granules described, beet bulk is also recommended as a diet addition for human food “for gastric regulation and cholesterol-conscious nutrition”. Beet ballast material is produced by sugar refineries. The substance can be obtained directly at the mill or in health food centers. It is not available only from pharmacists or on prescription, that is it is not presently classified as a therapeutic agent or medicament.

The administration of the beet bulk material preferably takes place in the form of granules, powders or cookies. The administration form as granules, in particular as sugar-coated granules, is preferred in order to facilitate swallowing of the relatively dry beet bulk material in the powder state by the patient.

In the context of the present invention, doses on the administration of the beet bulk material, in particular as sugar-coated granules, in individual doses of between approximately 5 g to approximately 20 g have proven advantageous. Individual doses of between approximately 10 g and approximately 20 g are preferred. Preferably, the taking of the beet bulk material takes place before or at the beginning of a meal.

By means of the investigations in the context of the present invention, it was possible to show that for the therapy, in particular of the early stages of diabetes mellitus type II, a combination of the active compound beet bulk material with a further active compound selected from acarbose, miglitol or another alpha-glucosidase inhibitor leads to particularly good results both with respect to the improved therapy action by the active compound combination and with respect to significantly reduced side effects in comparison with an administration of the individual active compounds.

The formula for acarbose is shown below:

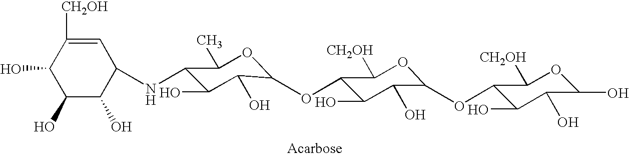

The formula for miglitol is shown below:

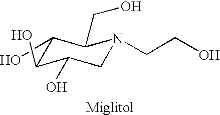

In addition to acarbose and miglitol, for example, voglibose or emiglitate are suitable as alpha-glucosidase inhibitors.

The administration of such a combination according to the invention can take place as a mixed preparation which simultaneously contains beet bulk material and/or acarbose, miglitol or other alpha-glucosidase inhibitors. The separate taking of beet bulk material and acarbose or miglitol is just as easily possible. The taking of both medicaments should if possible take place in a direct timewise relationship. In these cases also, the beet bulk material can be administered in a different administration form, taking as granules, in particular as sugar-coated granules, being preferred. The taking of the second active compound acarbose and/or miglitol or of another alpha-glucosidase inhibitor can be carried out, for example, in tablet form.

As a rule, the amount of the active compound acarbose/miglitol in the medicament or in the medicament combination according to the invention is significantly lower than the amount of the beet bulk material taken. Since, for example, amounts of 5 to 30 g, preferably approximately 10 to approximately 20 g, of beet bulk material are regarded as particularly suitable and amounts of approximately 20 to approximately 200 mg of acarbose and/or miglitol, preferably amounts of approximately 50 mg to approximately 100 mg of acarbose/miglitol per dose, the amount of beet bulk material is as a rule approximately 25-fold to 1000-fold the amount of acarbose or miglitol, quantitative ratios of approximately 400-fold to 100-fold of beet bulk material based on the other active compound(s) being given in the preferred ranges.

It was possible to investigate further bulk materials and alpha-glucosidase inhibitors, significant knowledge about the active constituents of the bulk materials being obtained with respect to use for the treatment of diabetes mellitus.

In the context of a variant of the invention, the combination of plant bulk materials with alpha-glucosidase inhibitors has in particular proven advantageous.

In the context of a refinement of the invention, the use of at least one bulk material having a high proportion of insoluble fiber materials is particularly preferred. The proportion of insoluble fiber materials which is contained in such a bulk material can be, for example, more than 20 g per 100 g of bulk material.

In the context of a preferred refinement of the invention, the individual dose to be administered preferably contains approximately 2.5 to 10 g of insoluble fibers. The taking of the composition according to the invention with the main meals is particularly preferred, for example 1 to 3 times daily.

Preferably, the taking of a composition according to the invention is carried out in combination with one of the alpha-glucosidase inhibitors acarbose, miglitol, voglibose or emiglitate or similar substances having comparable properties and similar chemical constitution. The taking of the bulk material in combination with at least one of the alpha-glucosidase inhibitors mentioned or immediately before or after its taking is particularly preferred.

The investigations carried out in the context of the present invention led, inter alia, to the discovery that other plant bulk materials exhibit a similar hypoglycemic action to beet bulk material, and that some bulk materials, similarly to beet bulk material, enhance the action of alpha-glucosidase inhibitors. Linseed, for example, showed a good action here, it being observed, however, that a regular effect with linseed on its own can be demonstrated only after defatting. It was furthermore observed that some bulk materials do not show the hypoglycemic action mentioned in combination with certain alpha-glucosidase inhibitors.

Extensive further experiments of the applicants have led to the surprising discovery that the taking of a cellulose-containing preparation, which preferably contains the cellulose in pure form without the other substances contained in the beet bulk material or other plant bulk materials, leads to particularly good results in the treatment of diabetes. As a result of these experiments, it can be assumed therefrom that the antidiabetic effect is to be attributed to the cellulose, which is also contained in insoluble bulk material-fiber mixtures of the type mentioned beforehand.

In contrast to the beet bulk material mentioned beforehand, cellulose is an almost neutral-tasting agent, which can be swallowed easily, administered for example, in powder form in water, yoghurt or similar foodstuffs and, as the experiments of the applicants have shown, has no side effects or undesired concomitant actions. Taking is therefore even simpler for the patient than in the case of beet bulk material preparations.

The preparation used for the composition according to the invention can contain more or less pure cellulose, which can be prepared inexpensively and is readily available. Since the hypoglycemic action in the bulk material preparations mentioned at the outset is obviously also to be attributed to the cellulose, the further advantage results that when taking pure cellulose the taking of a smaller amount of the composition suffices. The antidiabetic action of the cellulose is similar to that of the beet bulk or of the defatted linseed investigated beforehand. A considerable advantage of the cellulose, however, lies in the fact that it can be administered in chemically pure form and thus its easier standardizability as a medicament is afforded and moreover a smaller amount of substance suffices as a therapeutic dose. The efficacious individual dose of the cellulose can be reduced in comparison with the more complex bulk materials mentioned obtained from plants to, for example, approximately half to approximately one-tenth of the amount of substance. This facilitates use in diabetes therapy. Cellulose enhances the antidiabetic effect of the alpha-glucosidase inhibitors.

A further advantage of the use of cellulose in the composition according to the invention lies in the fact that it is a substance which is present in many foodstuffs and is therefore completely harmless for the human body. The cellulose can be formulated, for example, as a powder or alternatively as a chewable tablet in combination with a binder. Since cellulose powder can be suspended very readily and rapidly in customary foodstuff liquids, the taking of cellulose powder is convenient for the patient and the handling is very simple. The preferred individual doses mentioned correspond to approximately the amount of one tablespoon. This amount can be added to a drink and stirred in and thus administered conveniently for the patient. The composition according to the invention can, for example, be formulated such that the amount of cellulose powder in each case necessary for an individual dose reaches the market, for example, packed in a separate sachet on the one hand and the alpha-glucosidase inhibitor in tablet form on the other hand, in a common relatively large pack unit.

Investigations in the context of the present invention have shown that in principle, instead of the use of cellulose, the taking of a cellulose derivative is also suitable. Such cellulose derivatives can, for example, be substituted celluloses, which are optionally not of plant origin, but are prepared synthetically. By way of example, methylcellulose may be mentioned here, which is suitable in the context of the present invention as a bulk material instead of cellulose. Such a cellulose derivative is also preferably combined with an alpha-glucosidase inhibitor, it being possible for both substances to be present in combination in one preparation or in each case in separate preparations.

In the context of the present invention, defatted linseed has likewise proven suitable for the therapy of diabetes mellitus.

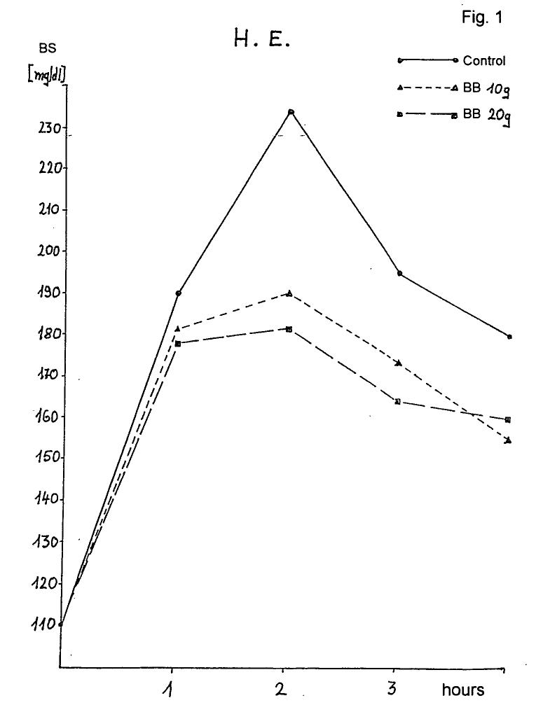

The present invention is described in more detail below with the aid of working examples. Reference is made here in the following text to the tables 1 to 7 attached in the appendix and to the FIGS. 1 to 8 likewise attached. A legend with explanations for FIGS. 1 to 8 is likewise attached to the application text as an annex.

We tested sugar beet bulk material having a high content of cellulose and hemicellulose and relatively little lignin (ratio C:H:L=6:6:1), linseed having a high content of hemicellulose (ratio C:H:L=2.5:5:1), wheat bran having the highest lignin content (ratio C:H:L=2.6:2.7:1), pure cellulose and, as a comparative substance, methyl-cellulose, a synthetic cellulose derivative, which in contrast to cellulose has a low water solubility and in this respect stands between the insoluble and insoluble bulk materials. The composition of the bulk material preparations can be seen in table 1.

Beet bulk material (Hammermühle Diät GmbH, D-67489 Kirrweiler) is granules (fine-grain chips) which taste unpleasant suspended in water, but can be swallowed readily in yoghurt. By sugar-coating the granules, an even better taking quality is achieved.

Linseed (commercially available in a health food center) is used as coarse granules (“rough-ground linseed”) and is defatted, for example, with acetone. It can be taken with water or more pleasantly in yoghurt and tastes nut-like.

Wheat bran (commercially available in a health food center) is a flocculent powder which is taken like linseed.

Cellulose (Synopharm GmbH, Postfach 1205, D-22882 Barsbüttel) in the preparation used is a fine mealy powder, which also tastes mealy and can easily be taken suspended in water or yoghurt.

Methylcellulose (Synopharm GmbH, Postfach D-22882 Barsbüttel) is likewise a mealy powder, which masses together to give sticky lumps in water or yoghurt and is less pleasant to take than cellulose.

### Short-Term Experiments with Bulk Materials

After measurement of the fasting blood sugar in the morning, 20 type II diabetics received a test breakfast (75 g of white bread, 20 g of butter, 40 g of jam or, as a sugar-free variant, 75 g of white bread, 20 g of butter and 40 g of cheese). On the control days, before the test breakfast 75 ml of yoghurt were given, and on the test days the various bulk materials were given in 75 ml of yoghurt. The bulk materials amounted to 10-20 g (beet bulk material, linseed and wheat bran) or 3-10 g (cellulose and methylcellulose). After the test breakfast, the blood sugar values were measured at ½ to 1 hour intervals for 2-4 hours. Each short-term experiment was carried out two to three times with the individual subjects. The respective average values are indicated. On the mornings of the experiment, the patients, provided that they had otherwise been treated antidiabetically, left out their corresponding medicaments (insulin, sulfonylurea or biguanide preparation).

The basis for the evaluation is the graphic representation of the blood sugar values as curves. The increasing phase of the blood sugar lasting 1-2 hours is determined planimetrically and considered as a measure of the lower limit of glucose absorption. In the first hour after the test breakfast, under physical quasi-resting conditions the increase in the blood sugar curve ought to reflect the intestinal glucose absorption into the blood almost unadulteratedly on the morning of the experiment in the diabetic differently from in the healthy person. Beyond the first hour, even in most type II diabetics an insulin response commences more or less clearly which levels off the blood sugar increase, or conversely in any case leads to a corresponding underestimation of the glucose absorption. In isolated cases, the control curve reaches the peak earlier than the experimental curves. The planimetric evaluation of all curves is then extended until achieving the peak of the last curve. This leads to the fact that the experimental hypoglycemias determined are underestimated rather than overestimated. For practical reasons, we compare the increasing phases of the blood sugar curves under the various experimental conditions (control, after bulk material, in the later experiments also after alpha-glucosidase inhibitors) with one another and calculate from this the degree of hypoglycemia based on the control curve. The lowering of the increase in blood sugar observed after treatment with bulk materials or other substances thus corresponds, under the assumption of the time-constant insulin response in the same patient, to the lowering of glucose absorption achieved. The statistical evaluation, which is possible only in the short-term experiments on account of the amounts of data, is carried out using the student's t test in a paired comparison.

Typical examples for beet bulk material, linseed and cellulose are shown in FIGS. 1-3. These bulk materials lowered the blood sugar increase after a test meal with a certain variation breadth regularly, on average by 30% (table 2). The lowering is highly significant (p<0.001). A difference in action between beet bulk material, linseed and cellulose cannot be detected. On the other hand, wheat bran is not markedly active (blood sugar lowering by approximately 10%). In a number of cases, various doses of the bulk materials were tested: A halving of the 20 g dose of beet bulk or linseed additionally afforded an 80-90% action. In the case of cellulose, 5, 6 and 7 g acted virtually identically. Three and 10 g achieved approximately 80% of the maximum action.

### Long-Term Experiments with Bulk Materials

Before the three main meals, 11 type II diabetics in each case received 10 g of beet bulk material or 5 g of cellulose. The blood sugar was determined daily 2 hours after individual breakfast, in most cases also the fasting blood sugar. A bulk material-free observation phase before the treatment period is considered as a control. In the case of a pretreatment, the antidiabetic medicaments were maintained unchanged during the addition of bulk material.

Table 3 summarizes the results: After exclusive or supplementary therapy with bulk materials, on average the fasting blood sugar was lowered by 4 mg/dl and the blood sugar after eating was lowered by 35 mg/dl. In two patients, whom it was possible to observe for sufficiently long (4 months), the HbA1C value decreased by 0.6 and 0.9% respectively.

### Side-Effects of the Bulk Materials

Undesired concomitant effects did not occur in the short-term experiments and occurred in 2 of the 11 patients during the long-term experiments, in each case with beet ballast material: In one patient each morning (not at midday and in the evening) after taking beet bulk material a 1-hour heartburn occurred, in another patient the feces rate doubled from 2 to 4 times daily.

### Composition and Evaluation of the Bulk Material Experiments

Water-insoluble ballast materials have a clear and in each individual case reproducible antidiabetic action. The active bulk fiber is cellulose. Obviously, the accompanying bulk fibers hemicellulose and lignin play a subordinate role. Why wheat bran only has a very poor action in spite of a high content of cellulose is unclear. Probably, accompanying substances, e.g. the abundantly contained protein (15%), abolish the antidiabetic action of the cellulose. Methylcellulose also obviously has only a weak effect. Since insoluble bulk materials such as cellulose are not cleaved and absorbed in the small intestine and their hypoglycemic effect commences in spite of this within 1 hour, they must prevent the glucose absorption within the intestine. This probably takes place by means of a partial inhibition of the starch-cleaving amylases, which is favored by the great chemical similarity of cellulose (beta-1,4-glycosidic glucose polymer) and amylose (alpha-1,4-glycosidic glucose polymer). Further findings in our short-term experiments support this assumption: cellulose did not have a hypoglycemic action after administration of pure sucrose (which is cleaved and absorbed independently of the amylase).

The hypoglycemic effect of the cellulose and the other bulk material preparations investigated is relatively low at on average 30%. The bulk materials are therefore suitable as monotherapy only for the treatment of the early stages of type II diabetes. In the other stages of the disease, they can be combined with other antidiabetics.

### Combination with Alpha-Glucosidase Inhibitors

The alpha-glucosidase inhibitors known hitherto are sugar-like substances which competitively inhibit the alpha-glucosidases in the small intestine and thus slow down the cleavage of the disaccharides contained in the food and the oligosaccharides resulting during starch degradation. We use the substances acarbose (Glucobay®) and miglitol (Diastabol®) hitherto permitted for therapy. Still further alpha-glycosidase inhibitors have been developed, e.g. voglibose and emiglitate, which all have an identical hypoglycemic action in principle. It is tested here whether insoluble bulk materials modify the hypoglycemic effect of acarbose and miglitol.

### Short-Term Experiments with Alpha-Glucosidase Inhibitors and Bulk Materials

21 patients were investigated: 10 each received acarbose and miglitol, 1 patient first acarbose and then miglitol. The individual dose used was 25-100 mg. The bulk materials used were beet bulk (10-20 g), defatted linseed (8-15 g), cellulose (3-10 g) and methylcellulose (5-6 g).

Immediately before the test meals described above, the subjects received either a) 75 ml of yoghurt (control experiment) or b) bulk material in yoghurt (bulk curve) or c) a tablet of acarbose or miglitol and then 75 ml of yoghurt (alpha-GI curve) or d) this medicament 5 minutes before taking bulk material in yoghurt (bulk-plus alpha-GI curve). As described, the blood sugar values were determined before and for 3 hours after the test meal.

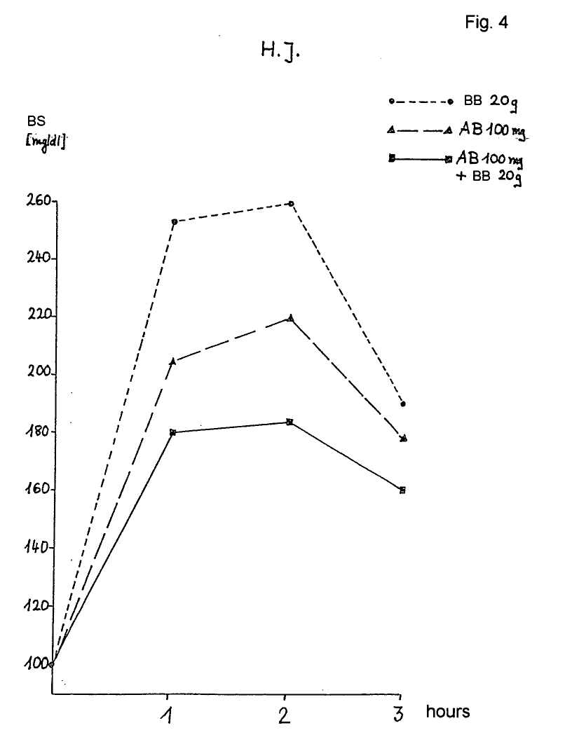

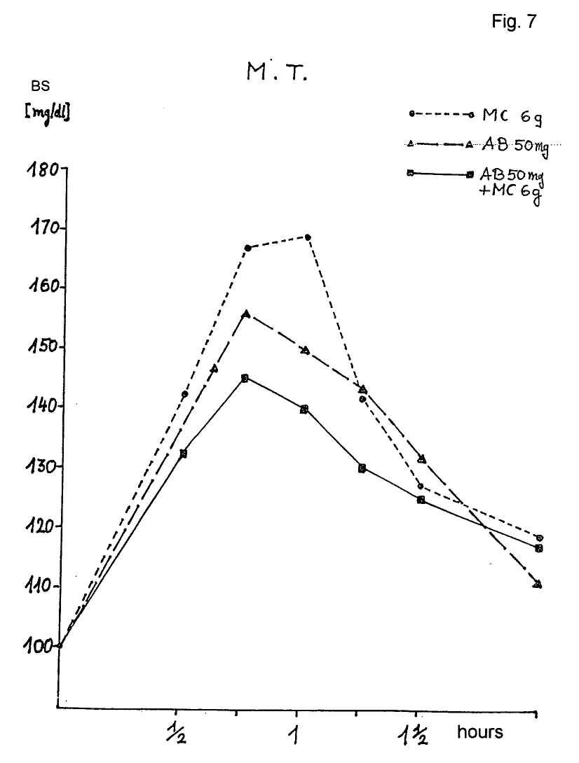

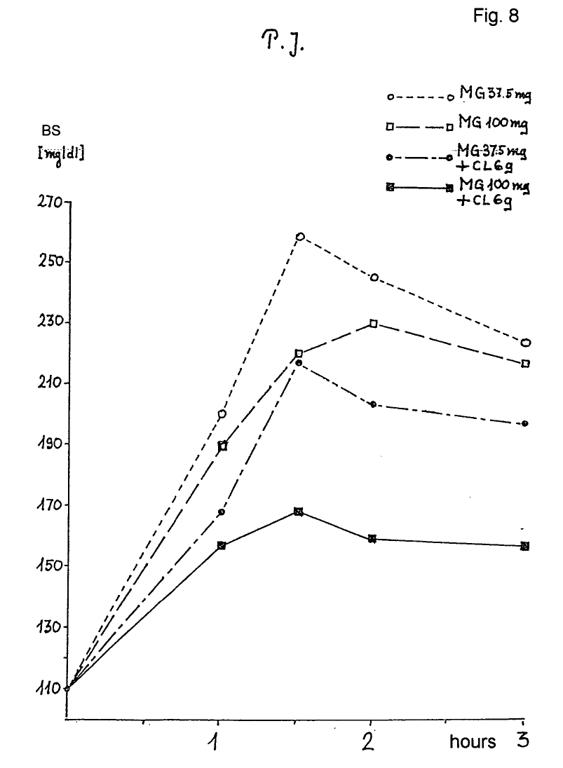

Typical examples are documented in FIGS. 4 to 7. Acarbose and miglitol clearly levelled off the blood sugar increase after eating in a known manner. They had a considerably stronger action than bulk material. A combination of glucosidase inhibitors and bulk material increased the effect of the two individual substances relevantly. This pattern was to be observed with quantitative variations in 24 of the 25 series of experiments carried out. The enhancement in action of the combination compared with the individual substances was highly significant (table 2). On average, the lowering of the postprandial blood sugar increase was 30.0+/−8.9% by bulk material on its own (a), 48.6+/−17.9% by alpha-glucosidase inhibitor (b) and 66.8+/−17.9% by the combination (c). The differences between (a) and (c) and also between (b) and (c) are statistically significant with an error probability of in each case p<0.001. The superiority of the combination is even more clear if the bandwidth of the action is investigated (table 4): A more than 50% lowering of blood sugar was to be observed with the glucosidase inhibitors only in 18 of 35 cases, but with the combination in 33 of the 35 cases. A lowering by more than 80% was not recorded with the glucosidase inhibitors in any case, but with the combination in 7 of the 35 experiments. With miglitol, in addition to the recommended standard dose of 100 mg the dose 25 mg was also investigated in 6 cases and the dose 50 mg was investigated 8 times (table 2, FIG. 8). The enhancement in action of the miglitol by a fixed dose of bulk material occurred at each dose level. By combination with bulk material, the action of 25 mg of miglitol was raised to a level of 100 mg without accompanying bulk material (table 5). The combination with the low dose lowered the blood sugar increase by on average 56%, the four-fold higher dose on its own brought about a lowering of 58%. In some cases, various acarbose doses were also investigated. Here, 25 mg plus bulk material only had a somewhat weaker action than 100 mg on its own, and 50 mg plus bulk material surpassed the effect of 100 mg on its own. Differences between the bulk materials cellulose, beet bulk and defatted linseed in the enhancement of action of the glucosidase inhibitors are not discernible (table 2). Only in the case of the methylcellulose not mentioned in the table does the enhancement effect appear to be somewhat lower (FIG. 7).

### Long-Term Experiments with Alpha-Glucosidase Inhibitors and Bulk Materials

In 6 patients, during the experimental period the blood sugar was measured and recorded daily according to protocol independently in the morning in the fasting state and 2 hours after individual breakfast. In 2 patients treated over a half-yearly period, the HbA1C value and, as a spot check, the 1-hour value of the blood sugar were also determined every month after breakfast. In a control phase lasting a number of weeks with maintenance of the premedication the test phases took place in stages lasting a number of weeks with the additions of bulk material and glucosidase inhibitor. Before breakfast, midday and evening meal, first acarbose 25-100 mg or miglitol (25-50 mg) and then beet bulk material (10 g) or cellulose (5 g) were taken.

Table 6 summarizes the results. In each individual case, bulk material and acrabose/miglitol increased in combination. On average, the fasting blood sugar fell by 17 mg/dl after 60 days combination treatment and the blood sugar increase after eating fell by 49 mg/dl.

This 2-hour value probably only inadequately represents the improvement in the metabolic position achieved, because (as known from the short-term experiments) the postprandial hypoglycemias after 1 hour are more representative than after 2 hours. In the half-yearly observations of 2 otherwise untreated patients mentioned, the blood sugar increase decreased 1 hour after breakfast by finally 69 and 91 mg/dl respectively, and the HbA1C values decreased by 1.3% (table 7).

### Side Effects of the Combination Treatment

In the short-term experiments with 100 mg of acrabose on its own, 3 of 9 patients indicated abdominal symptoms lasting for a number of hours. In the case of miglitol, with 4 of 9 patients semiliquid stools as far as diarrhea occurred. With 50 mg of acarbose (2 cases) and 50 mg of miglitol (8 cases) or 25 mg of miglitol (5 cases), no clear concomitant symptoms were to be observed. With all combinations of bulk material with acarbose or miglitol, even with the 100 mg doses, no side effects occurred in the short-term experiments. If normal persons received 50-75 g of sucrose instead of the test breakfast, abdominal pain and diarrhea occurred after 100 mg of acarbose or miglitol. These symptoms were markedly alleviated on combination of the glucosidase inhibitor with beet bulk material or with cellulose. In the long-term observations, 4 of the 6 patients indicated abdominal symptoms and flatulence with the glucosidase inhibitors, particularly after 100 mg 3 times daily. By addition of beet bulk or cellulose, the symptoms passed, but were present in mild form in one patient on 50 mg of miglitol 3 times for a further 14 days. In supplementary investigations, every 5 subjects received 50 mg of acarbose or miglitol plus 5 g of cellulose 3 times for two days. No undesired symptoms occurred.

Why insoluble bulk fibers reduce intestinal concomitant effects of the alpha-glucosidase inhibitors can only be presumed: On the one hand, insoluble bulk fibers reduce the symptoms of increased intestinal motoricity, on the other hand due to the inhibition of amylase fewer disaccharides are formed, which would lead to irritation in the large intestine.

### Composition and Evaluation of the Combination Experiments

By the combination of alpha-glucosidase inhibitors and insoluble bulk materials, a significant advance in the therapy of type II diabetes is achieved. The action of the glucosidase inhibitors is increased by more than a third, so that the blood sugar increase after eating is reduced on average by almost 70%. This means in the individual case, for example in a diabetic having a fasting blood sugar of 120 mg/dl and a maximum post-prandial blood sugar increase to 220 mg/dl, that this is lowered by the combination therapy to 150 mg/dl. This is a value in the upper normal range.

Since the hypoglycemic effect is based on an inhibition of absorption of carbohydrates in the small intestine, the diabetic treated in this manner saves insulin, his insulin production recovers, and the insulin resistance decreases, as is discernible in the long-term experiments by the decreasing fasting sugar in the morning. No relevant side effects (as with the other antidiabetics) oppose this extremely positive influence on the course of the disease. The frequency of undesired concomitant effects with glucosidase therapy on its own decreases with the combination from approximately 50% to 5%. On account of the dose reduction from 100 to 50 mg which is possible in most cases, the intensity of these concomitant symptoms is additionally attenuated, such that they are tolerated without problems by the few patients affected.

From the point of view of therapeutic practicability, cellulose is the ideal bulk material. It is available as a pure substance, easily suspendable in liquids, neutral in taste, effective in amounts of 3-7 g which can be swallowed without problems and on top of everything good value. According to the unanimous opinion of leading diabetologists, a Europe and in the USA approximately half of type II diabetics are inadequately adjusted with a tablet therapy. With the novel therapy according to the invention, most of these patients could be optimally treated.

TABLE 1
Bulk material content per 100 g of the bulk material preparations
investigated (according to manufacturer's instructions)*

| | Cellulose | | Hemicellulose | Lignin | Pectin |
|---|---|---|---|---|---|
| Beet | 24 | g | 24 g | 4 g | 31 g |
| bulk | | | | | |
| Linseed** | 7 | g | 13 g | 3 g | 24 g |
| Wheat bran | 21 | g | 22 g | 8 g | — |
| Cellulose | 99 | g | — | — | — |
| Methyl- | (99 | g)*** | — | — | — |
| cellulose | | | | | |

*The remainder consists of protein, fat, sugar and minerals
**By acetone treatment, the fat content of the linseed chips used was reduced from 40 to 14%.
***Synthetically modified cellulose

TABLE 2
Decrease in the blood sugar increase after a test breakfast
by the bulk materials beet bulk (BB), linseed (LS)
and cellulose (CL) or/and the alpha-glucosidase inhibitors
acarbose (AB) and miglitol (MG)

| | | | Hypoglycemia | | |
|---|---|---|---|---|---|
| | Treatment | | | Alpha- | Bulk + |
| Patient | Bulk | Alpha-GI | Bulk | GI | alpha-GI |
| H. J. | BB 20 g | AB 100 mg | 18% | 41% | 57% |
| S. W. (n*) | BB 20 g | AB 100 mg | 40% | 63% | 81% |
| S. W. (sf**) | BB 20 g | AB 100 mg | 45% | 56% | 78% |
| T. W. | BB 20 g | AB 100 mg | 46% | 65% | 84% |
| J. K. (n*) | BB 20 g | AB 100 mg | 4% | 6% | 65% |
| J. K. (sf**) | BB 20 g | AB 100 mg | 27% | 55% | 60% |
| F. J. | BB 20 g | AB 100 mg | 17% | 39% | 60% |
| H. E. | BB 20 g | AB 50 mg | 31% | 42% | 50% |
| F. J. | BB 20 g | MG 100 mg | 19% | 24% | 53% |
| S. A. | BB 20 g | MG 100 mg | 40% | 60% | 82% |
| W. U. | LS 15 g | AB 100 mg | 28% | 63% | 68% |
| S. W. | LS 15 g | AB 100 mg | 51% | 58% | 60% |
| K. F. | LS 15 g | MG 50 mg | 24% | 33% | 42% |
| S. A. | LS 15 g | MG 25 mg | 46% | 27% | 68% |
| S. A. | LS 15 g | MG 100 mg | 46% | 80% | 90% |
| A. C. | CL 6 g | AB 50 mg | 28% | 50% | 76% |
| A. C. | CL 6 g | AB 100 mg | 28% | 59% | 82% |
| S. M. | CL 6 g | MG 50 mg | 19% | 40% | 75% |
| W. H. | CL 6 g | MG 50 mg | 17% | 37% | 50% |
| M. S. | CL 6 g | MG 50 mg | 28% | 62% | 73% |
| S. W. | CL 7 g | MG 25 mg | 33% | 47% | 70% |
| S. W. | CL 7 g | MG 50 mg | 33% | 62% | 74% |
| S. W. | CL 7 g | MG 100 mg | 33% | 59% | 93% |
| P. J. | CL 6 g | MG 25 mg | 22% | 12% | 35% |
| P. J. | CL 6 g | MG 50 mg | 22% | 25% | 57% |
| P. J. | CL 6 g | MG 100 mg | 22% | 32% | 64% |
| B. A. | CL 6 g | MG 25 mg | 36% | 53% | 60% |
| B. A. | CL 6 g | MG 50 mg | 36% | 59% | 69% |
| B. A. | CL 6 g | MG 100 mg | 36% | 66% | 74% |
| S. A. | CL 6 g | MG 25 mg | 30% | 49% | 70% |
| S. A. | CL 6 g | MG 100 mg | 30% | 75% | 89% |
| F. J. | CL 6 g | MG 25 mg | 27% | 24% | 35% |
| F. J. | CL 6 g | MG 100 mg | 27% | 34% | 63% |
| B. W. | CL 6 g | MG 50 mg | 30% | 71% | 70% |
| B. W. | CL 6 g | MG 100 mg | 30% | 73% | 58% |
| Mean values | | | 30.0% ± | 48.6% ± | 66.8% ± |
| | | | 8.9 | 17.9 | 15.0 |

*n = normal test breakfast
**sf = sugar-free test breakfast

TABLE 3
Long-term effect of beet bulk material and cellulose

| | | | | | after BM | | |
|---|---|---|---|---|---|---|---|
| | Con- | before BM | | | | Blood sugar | |
| | comitant | (control) | | | Time | (mg/dl) | |
| Patient | therapy | FBS** | ppBS*** | BM | (days) | FBS | ppBS |
| K. P. | — | — | 135 | BB | 120 | — | 104 |
| B. R. | — | — | 159 | BB | 120 | — | 105 |
| H. E. | — | 145 | 164 | BB | 13 | 137 | 138 |
| S. E. | — | 122 | 181 | BB | 22 | 120 | 151 |
| G. W. | GB, MF | 139 | 175 | BB | 28 | 134 | 110 |
| G. T. | GP | — | 161 | BB | 14 | — | 106 |
| P. E. | GP | 133 | 155 | BB | 15 | 131 | 119 |
| H. J. | insulin | 164 | 216 | BB | 10 | 158 | 192 |
| S. H. | GP, MF | 181 | 200 | BB | 7 | 182 | 177 |
| L. S. | — | 140 | 185 | CL | 14 | 136 | 170 |
| G. R. | MG | 118 | 175 | CL | 42 | 116 | 148 |

| | | | | |
|---|---|---|---|---|
| Mean values | 143 | 173 | 139 | 138 |

*BM = bulk material
**FBS = fasting blood sugar
***ppBS = blood sugar 2 hours after breakfast
GB = glibornuride
GP = glimepiride
MF = metformin
MG = miglitol
BB = beet bulk material
CL = cellulose

TABLE 4
Effectiveness of the alpha-glucosidase inhibitors. Comparison
of monotherapy with combination therapy of glucosidase inhibitors
plus bulk material. Results of 35 short-term experiments.

| Lowering of the | | |
|---|---|---|
| postprandial | | |
| blood sugar | Alpha-GI* | Alpha-GI plus BM** |
| >40% | 23 | 34 |
| >50% | 18 | 33 |
| >60% | 10 | 23 |
| >70% | 4 | 13 |
| >80% | 0 | 7 |

*Alpha-GI = alpha-glucosidase inhibitor
**BM = bulk material

TABLE 5
Comparison of action of miglitol 25 mg plus
bulk material and miglitol 100 mg on its own

Hypoglycemia

| Patient | Bulk material | MG 25 plus BM | MG 100 |
|---|---|---|---|
| S. A. | LS | 68% | 80% |
| S. W. | CL | 70% | 59% |
| P. J. | CL | 35% | 32% |
| B. A. | CL | 60% | 66% |
| S. A. | CL | 70% | 75% |
| F. J. | CL | 35% | 34% |
| Mean values | | 56.3% | 57.7% |

MG = miglitol
BM = bulk material
LS = linseed
CL = cellulose

TABLE 6
Long-term effect of the combination of bulk
material with alpha-glucosidase inhibitors

| | Con- | | | | BM plus alpha-GI | | | |
|---|---|---|---|---|---|---|---|---|
| | comitant | Control | | | | Time | | |
| Pat. | Therapy | nBS | ppBS | BM | Alpha-GI | (days) | nBS* | ppBS** |
| H. J. | Insulin | 164 | 216 | BB 10 g | AB 100 | 14 | 151 | 167 |
| P. E. | GP | 133 | 155 | BB 10 g | AB 50 | 14 | 130 | 107 |
| S. H. | GP, MF | 187 | 200 | BB 10 g | AB 50 | 8 | 166 | 162 |
| | | 187 | 200 | BB 10 g | AB 100 | 14 | 147 | 142 |
| G. R. | — | 118 | 175 | CL 5 g | MG 50 | 42 | 116 | 148 |
| L. S. | — | 140 | 185 | CL 5 g | MG 50 | 150 | 115 | 126 |
| A. C. | — | 128 | 168 | CL 5 g | AB 25 | 178 | 110 | 104 |

| | | | | | |
|---|---|---|---|---|---|
| Mean values | 151 | 186 | 60 | 134 | 137 |

*nBS = fasting blood sugar (mg/dl)
**ppBS = blood sugar 2 hours after breakfast (mg/dl)
BM = bulk material
Alpha-GI = alpha-glucosidase inhibitor
GP = glimepiride
MF = metformin
BB = beet bulk material
CL = cellulose
AB 50, 100 = acarbose 50 and 100 mg
MG 25, 50 = miglitol 25 and 50 mg

TABLE 7
Six-month long-term therapy with cellulose
(CL) plus acarbose (AB) or miglitol (MG)

| | A.C.* | | L.S.** | |
|---|---|---|---|---|
| | ppBS*** | HbA1C | ppBS*** | HbA1C |
| | (mg/dl) | (%) | (mg/dl) | (%) |
| before therapy | 194 | 7.1 | 222 | 7.9 |
| after 1 month | 140 | 6.9 | 161 | 7.8 |
| after 3 months | 132 | 6.1 | 146 | 7.0 |
| after 5-6 months | 125 | 5.8 | 131 | 6.6 |

*3 × 25 mg AB + CL (6 months)
**3 × 50 mg MG + CL (5.5 months)
***postprandial blood sugar 1 hour after breakfast (mean value of 3 days)

### Legends to FIGS. 1-8

FIG. 1: Beet bulk material effect. H.E., diabetes type II, 64 years, otherwise treated with glimepiride (sulfonylurea preparation). Blood sugar curve after test breakfast and 10 or 20 g of beet bulk material (BB). Control curve without bulk material. The fasting blood sugar is at 110 mg/dl (measured values 106-115 mg/dl).

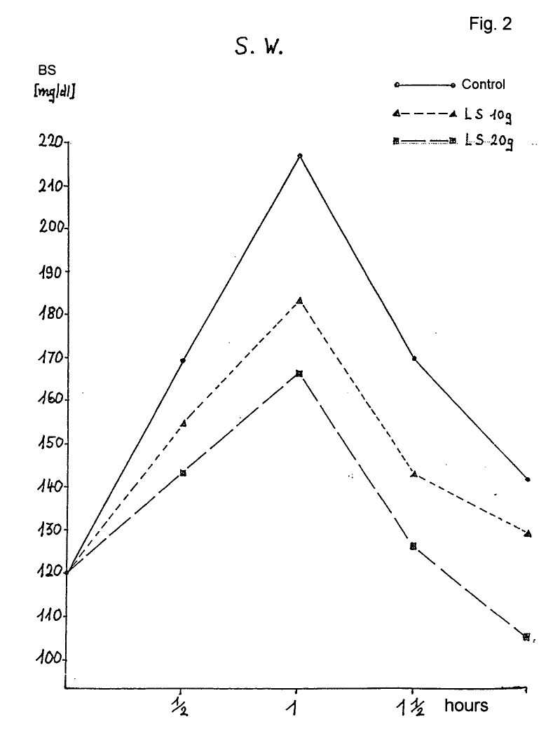

FIG. 2: Linseed effect.

S.W., diabetes type II, 67 years, untreated. Blood sugar curves after test breakfast and 10 or 20 g of defatted linseed (LS). The fasting blood sugar is standardized at 120 mg/dl (measured values 112-124 mg/dl)

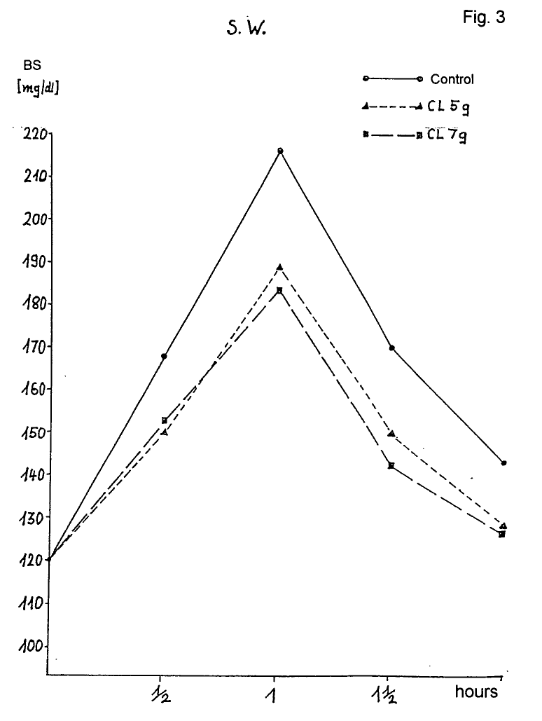

FIG. 3: Cellulose effect. S.W., diabetes type II, 67 years, untreated. Blood sugar curves after test breakfast and 5 or 7 g of cellulose (CL).

FIG. 4: Combination effect of beet bulk material and acarbose. H.J., diabetes type II, 59 years, otherwise treated with insulin. Blood sugar curves after 20 g of beet bulk (BB), 100 mg of acarbose (AB) and acarbose plus beet bulk (AB+BB). The fasting blood sugar is standardized at 100 mg/dl (measured values 98-114 mg/dl).

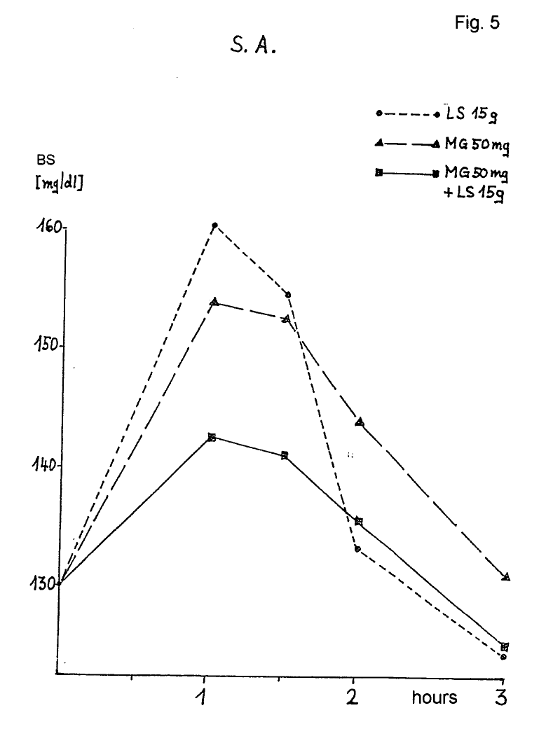

FIG. 5: Combination effect of linseed and miglitol. S.A., diabetes type II, 67 years, otherwise treated with glibenclamide. Blood sugar curves after 15 g of defatted linseed (LS), 50 mg of miglitol (MG) and miglitol plus linseed (MG+LS). The fasting blood sugar is standardized at 130 mg/dl (measured values 122-135 mg/dl).

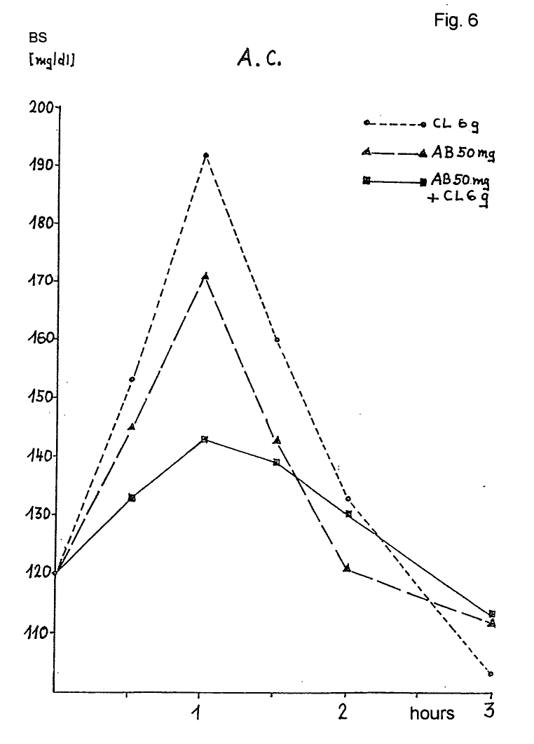

FIG. 6: Combination effect of cellulose and acarbose. A.C., diabetes type II, 60 years, untreated. Blood sugar curves after 6 g of cellulose (CL), 50 mg of acarbose (AB) and acarbose plus cellulose (AB+CL). The fasting blood sugar is standardized at 120 mg/dl (measured values 113-125 mg/dl).

FIG. 7: Combination effect of methylcellulose and acarbose. M.T., diabetes type II, 67 years, untreated. Blood sugar curves after 6 g of methylcellulose (MC), 50 mg of acarbose (AB) and acarbose plus methylcellulose (AB+MC). The fasting blood sugar is standardized at 100 mg/dl (measured values 100-113 mg/dl).

FIG. 8: Combination effect with various doses of a glucosidase inhibitor. P.J., diabetes type II, 59 years, otherwise treated with insulin. Blood sugar curves after individual doses of 37.5 mg or 100 mg of miglitol (MG) and after the combination of 37.5 mg of MG with 6 g of cellulose (MG 37.5 mg+CL) or 100 mg of miglitol with 6 g of cellulose (MG 100 mg+CL). The fasting blood sugar is standardized at 110 mg/dl (measured values 102-126 mg/dl).

1. A composition for the treatment of diabetes mellitus, characterized in that it comprises at least one plant bulk material in combination with at least one alpha-glucosidase inhibitor.

2. The composition as claimed in claim 1 for the treatment of diabetes mellitus type II.

3. The composition as claimed in claim 1 or 2, characterized in that the bulk material contains a high proportion of insoluble fiber materials.

4. The composition as claimed in one of claims 1 to 3, characterized in that the bulk material contains a proportion of preferably more than 20 g per 100 g of insoluble fiber materials.

5. The composition as claimed in one of claims 1 to 4, characterized in that it contains approximately 2.5 g to approximately 10 g of insoluble fibers per individual dose.

6. A composition for the treatment of diabetes mellitus, characterized in that it contains at least one plant bulk material for taking immediately before or after taking a composition containing an alpha-glucosidase inhibitor.

7. The composition as claimed in one of claims 1 to 6, characterized in that it contains at least one alpha-glucosidase inhibitor, preferably selected from acarbose, miglitol, voglibose and emiglitate.

8. The composition as claimed in one of claims 1 to 7, characterized in that it contains at least one alpha-glucosidase inhibitor in an amount of between approximately 25 and approximately 200 mg.

9. The composition for the treatment of diabetes mellitus as claimed in one of claims 1 to 8, characterized in that this contains at least one soluble bulk material and/or at least one insoluble bulk material.

10. The composition as claimed in claim 1 to 9, characterized in that this contains at least one soluble bulk material, at least one insoluble bulk material and at least one alpha-glucosidase inhibitor.

11. The composition as claimed in claim 1 or 10, characterized in that the soluble bulk material and/or the insoluble bulk material is a bulk material of plant origin.

12. The composition as claimed in one of claims 1 to 11, characterized in that at least one of the bulk materials contained is a plant bulk material which was obtained from a sacchariferous fruit.

13. The composition as claimed in one of claims 1 to 12, characterized in that at least one of the bulk materials is a bulk material which was obtained from a beet fruit.

14. The composition as claimed in one of claims 1 to 13, characterized in that at least one medicament containing beet bulk material is present in the form of granules, powder, cookies or as sugar-coated granules.

15. The composition as claimed in claim 14, characterized in that it comprises acarbose and/or miglitol or other alpha-glucosidase inhibitors, which are preferably present in tablet form.

16. The composition as claimed in one of claims 14 or 15, characterized in that this contains beet bulk material in an amount of approximately 5 g to approximately 30 g, preferably of approximately 10 g to approximately 20 g.

17. The composition as claimed in one of claims 1 to 13, characterized in that at least one of the bulk materials is a polysaccharide derivative.

18. The composition as claimed in one of claims 1 to 13 or 17, characterized in that this furthermore contains bulk materials or accompanying substances, in particular pectin and/or pentosan and/or lignin.

19. The composition as claimed in one of claims 1 to 13, 17 or 18, characterized in that at least one of the accompanying substances is a protein or protein derivative.

20. The composition as claimed in one of claims 1 to 13 or 17 to 19, characterized in that at least one alpha-glucosidase inhibitor is an oligosaccharide or a derivative thereof.

21. The composition as claimed in one of claims 1 to 13 or 17 to 20, characterized in that at least one alpha-glucosidase inhibitor is a monosaccharide, oligosaccharide or a derivative thereof containing an optionally substituted amino group.

22. A composition for the treatment of diabetes mellitus, characterized in that it comprises cellulose in combination with at least one alpha-glucosidase inhibitor.

23. The composition as claimed in claim 22, characterized in that the cellulose on the one hand and the alpha-glucosidase inhibitor on the other hand are present in separate preparations.

24. A composition for the treatment of diabetes mellitus, characterized in that it contains cellulose and is intended for taking after taking a composition containing an alpha-glucosidase inhibitor.

25. The composition as claimed in claim 22 or 24, characterized in that a cellulose-containing preparation is intended for taking in close timewise relationship after taking a composition containing an alpha-glucosidase inhibitor.

26. The composition as claimed in one of claims 22 to 25, characterized in that it is formulated as a combination preparation, where a first preparation contains cellulose and a second preparation contains at least one alpha-glucosidase inhibitor.

27. The composition as claimed in one of claims 22 to 26, characterized in that the cellulose is present in powder form.

28. The composition as claimed in one of claims 22 to 27, characterized in that the cellulose-containing preparation is formulated as a chewable tablet.

29. The composition as claimed in one of claims 22 to 28, characterized in that it contains an amount of approximately 1 to approximately 10 g of cellulose per dose.

30. The composition as claimed in one of claims 22 to 29, characterized in that it contains at least one alpha-glucosidase inhibitor in an amount of between approximately 10 and approximately 200 mg per dose.

31. The composition as claimed in one of claims 22 to 30, characterized in that it contains at least one alpha-glucosidase inhibitor, preferably selected from acarbose, miglitol, voglibose and emiglitate.

32. The composition as claimed in one of claims 22 to 31, characterized in that it contains an amount of approximately 3 to 7 g of cellulose per dose.

33. A composition for the treatment of diabetes mellitus, in particular of diabetes type II, characterized in that this comprises a cellulose derivative.

34. The composition as claimed in claim 33, characterized in that this contains a substituted cellulose, in particular methylcellulose, as a cellulose derivative.

35. The composition as claimed in claim 33 or 34, characterized in that this comprises a cellulose derivative and at least one alpha-glucosidase inhibitor, which can be present in combination in one preparation or in each case in separate preparations.

36. The composition as claimed in one of claims 1 to 35, characterized in that this contains defatted linseed as a bulk material.

37. The composition as claimed in one of claims 1 to 36, characterized in that at least the bulk material is intended for taking immediately before or at a meal.
